(19) United States
(12) Patent Application Publication
(10) Pub. No.: US 2026/0009704 A1
(43) Pub. Date: Jan. 8, 2026

(54) METHOD FOR MANUFACTURING STANDARD PARTICLES FOR QUALITY CONTROL IN TOTAL ORGANIC CARBON ANALYSIS AND STANDARD PARTICLES MANUFACTURED THEREBY

(71) Applicant: FOUNDATION FOR RESEARCH AND BUSINESS, SEOUL NATIONAL UNIVERSITY OF SCIENCE AND TECHNOLOGY, Seoul (KR)

(72) Inventors: Hyun Sang SHIN, Seoul (KR); Joo Won KIM, Daejeon (KR)

(73) Assignee: FOUNDATION FOR RESEARCH AND BUSINESS, SEOUL NATIONAL UNIVERSITY OF SCIENCE AND TECHNOLOGY, Seoul (KR)

(21) Appl. No.: 18/880,276

(22) PCT Filed: Jul. 5, 2023
(86) PCT No.: PCT/KR2023/009531

§ 371 Date: Dec. 31, 2024

(30) Foreign Application Priority Data
Jul. 5, 2022 (KR) 10-2022-0082324

(51) Int. Cl. G01N 1/40; G01N 1/28; G01N 1/42; G01N 33/18

(52) U.S. Cl. CPC G01N 1/4077; G01N 1/286; G01N 1/42; G01N 33/1846; G01N 2001/2866; G01N 2001/2893; G01N 2001/4083

(57) ABSTRACT

A method of preparing a standard particle for quality control in a total organic carbon analysis, includes: selecting and collecting a target sample of an environmentally similar matrix from an environmental sample, separating a supernatant from the target sample, cooling and freeze-drying a target sample, from which the supernatant has been separated, powdering the freeze-dried target sample by grinding, obtaining a homogenized particle sample by sieving the powdered target sample, and performing selection of a standard particle by evaluating the obtained particle sample.

### CROSS-REFERENCE TO PRIOR APPLICATIONS

This Application is a National Stage Patent Application of PCT International Application No. PCT/KR2023/009531 (filed on Jul. 5, 2023), which claims priority to Korean Patent Application No. 10-2022-0082324 (filed on Jul. 5, 2022), which are all hereby incorporated by reference in their entirety.

### ACKNOWLEDGEMENT

The Korean research and development project that supported this present disclosure is as follows.
Project Unique Number 1485019253
Project Serial Number 2020003030005
Government Department Ministry of Environment
Specialized Institution for Project Management Korea Environmental Industry and Technology Institute
Title of Research Business Aquatic Ecosystem Health Enhancement Technology Development Project
Title of Project Development of a source tracking tool for total organic carbon based on two dimensional fingerprinting analyses
Contribution Rate 1/2
Supervising Institute Se jong University Industry-Academia Cooperation Foundation
Research Period 2020 Apr. 22 to 2023 Dec. 31
Project Unique Number 1345366159
Project Serial Number 2020R1A6A1A03042742
Government Department Ministry of Education
Specialized Institution for Project Management National Research Foundation of Korea
Title of Research Business R&D Program for Science and Engineering Research Infrastructure
Title of Project Environmental Technology Research Institute
Contribution Rate 1/2
Supervising Institute Foundation for Research and Business, Seoul National University of Science and Technology
Research Period 2020 Jun. 1 to 2029 May 31

### BACKGROUND

The present disclosure relates to a method of preparing a standard particle for quality control in a total organic carbon analysis to provide a standard particle with versatility from actual environmental samples, and to the same standard particle prepared thereby.

Organic pollutant indicators such as biological oxygen demand (BOD) and chemical oxygen demand (COD) have been widely used for a long time as indicators to determine the impact of organic matter existing in water on an actual aquatic ecosystem.

However, recently non-point source pollutants and toxic substances have increased due to urbanization and industrialization, and non-degradable organic matter in the water system continues to increase due to eutrophication. Thus, conventional water quality management indicators centered on BOD and COD do not reflect the total amount of pollutants or their various properties.

Accordingly, under the Water Environment Conservation Act, the Ministry of Environment adopted total organic carbon (TOC) as an indicator for a test method to replace the conventional concept of chemical oxygen demand. As a result, starting in 2020, an organic matter management indicator for new public wastewater treatment facilities and wastewater discharge facilities shifted from chemical oxygen demand to total organic carbon. Starting in 2021, TOC has been applied as a water quality regulation item for public wastewater treatment facilities.

Under these developments, to evaluate the process efficiency of wastewater treatment facilities, it is important to accurately measure the total organic carbon in the wastewater influent and process stages. Therefore, unlike conventional public water-centered total organic carbon analysis, the demand for TOC measurement of water samples containing suspended particles of various properties is increasing.

However, the TOC measurement value can vary significantly depending on a sample matrix. In particular, the suspended particles of various properties contained in field samples not only cause incomplete oxidation but also cause clogging of device conduits or accumulation of solid materials in the oxidation column.

This is a major factor that reduces accuracy and reproducibility (i.e. precision) of the total organic carbon analysis. Since it is important to check appropriate sample homogenization pretreatment, instrument measurement mode, and condition settings, domestic and international official test methods (ES 04311.1c, ISO 8245, and EN 1484) stipulate separate quality control standards for samples containing suspended particles.

In most of these domestic and international official test methods, cellulose is used as a standard particle for quality control of total organic carbon in samples containing suspended particles. When analyzing actual environmental samples, to minimize the impact of suspended particles, it is suggested that “homogenization through ultrasonic waves or stirring is required”, and it is only stated that device setup and maintenance should follow the instructions of each device manufacturer.

However, cellulose is a pure compound and does not represent actual environmental samples of various properties. Unlike the actual environmental samples, cellulose has problems in that cellulose has a high oxidation rate and cannot be applied to quality control for a sample pretreatment process.

That is, due to those issues, cellulose has been simply used for quality control to ensure that the device setup is suitable for measuring suspended particles and that the device is suitable for handling particulate matter. However, cellulose has not been used for practical quality control verification for other sample handling and analytical procedures, which are important in handling samples containing suspended particles, including sample pretreatment.

Therefore, there is a need for the development of a standard particle of environmental origin, the standard particle being applicable to practical quality control in analytical procedures, including sample pretreatment, and to quality control of total organic carbon in samples containing suspended particles.

### SUMMARY

The objective of the present disclosure, which is derived from the aforementioned necessity, is to provide a method of preparing a standard particle for quality control in a total organic carbon analysis of water samples, wherein the water samples are of environmental origin, and contain suspended particles applicable to practical quality control, and to provide the same standard particle.

To achieve the objective, the technical gist of the present disclosure is to provide a method of preparing a standard particle for quality control in a total organic carbon analysis and the same standard particle prepared thereby, the method including: a step of selecting and collecting a target sample of an environmentally similar matrix from an environmental sample, a step of separating a supernatant from the target sample, a step of cooling and freeze-drying a target sample, from which the supernatant has been separated, a step of powdering the freeze-dried target sample by grinding, a step of obtaining a homogenized particle sample by sieving the powdered target sample, and a step of performing selection of a standard particle by evaluating the obtained particle sample.

Additionally, the standard particle may preferably have a particle size in a range of 100 μm or less.

Herein, the step of separating the supernatant may preferably involve a first supernatant separation process by precipitation and a second supernatant separation process by centrifugation.

Additionally, the step of cooling and freeze-drying may further involve a cooling process using liquid nitrogen.

Additionally, the step of obtaining the particle sample may preferably further involve mixing by shaking for 24 to 48 hours after the sieving.

Additionally, the step of obtaining the particle sample may preferably further involve a sterilization process through UV ray irradiation after the sieving.

Additionally, the step of performing selection of a standard particle may preferably involve evaluating homogeneity, stability, accuracy, and precision of the particle sample.

Additionally, it is preferable that, in the homogeneity evaluation, a between-bottle homogeneity (S_{b}) satisfies the condition of S_{b}≤ 0.3σ_{PA} when compared to the standard deviation (σ_{PA}) to be used in the proficiency evaluation, in which a standard deviation of 10% to 20% is applied in this range, making the between-bottle homogeneity (S_{b}) satisfy S_{b}≤3 to 6.

Additionally, in the step of performing selection of a standard particle, a recovery rate of 50% to 80% or more may preferably be set as an appropriate standard for selecting the standard particle.

Additionally, the homogeneity evaluation may be performed by applying a turbidity index (NTU₅/NTU₀) (in which the NTU₅/NTU₀ represents a turbidity ratio after 5 minutes compared to an initial turbidity at 0 minutes, following flotation).

The present disclosure is to provide a standard particle suitable for quality control of suspended particles when analyzing total organic carbon for water samples. To prepare a particle sample of environmental origin, target samples from an environmentally similar matrix are selected and collected, and the target samples are cooled, freeze-dried, and then homogenized. Thereby, the present disclosure provides a standard particle with versatility, which can be used for quality control for total organic carbon analysis of water samples containing suspended particles.

Thus, the standard particle according to the present disclosure can be used for reliable total organic carbon analysis procedures, condition settings, and proficiency evaluation such as sample handling, pretreatment, homogeneity evaluation, and device setup for water samples containing actual suspended particles. This allows practical quality control for an analysis procedure, including sample pretreatment, thereby improving a wastewater treatment process.

In addition, the standard particle according to one embodiment of the present disclosure is prepared from sewage sludge (excess sludge), which has completely undergone a biological-based wastewater treatment process, thereby the standard particle has the properties of particulate organic matter with low biodegradability, so the standard particle is highly stable and can be secured in large quantities. This creates a high added value of organic waste through recycling of excess sludge, making an economic effect.

In addition, it is generally important to accurately measure total organic carbon in wastewater influent in predicting an organic matter treatment rate for each unit process. By using the standard particle according to the present disclosure, the accurate measurement of total organic carbon for each unit process is possible. Thus, in the calculation of chemical dosage and processing time during the treatment process, a significant improvement can be achieved. Accordingly, the standard particle according to the present disclosure is expected to have a high potential for use in quality control to measure total organic carbon in wastewater treatment plants with a high possibility of inflow of suspended particles.

In addition, the present disclosure is expected to be highly useful for an accurate measurement evaluation of total organic carbon for river samples, which are affected by suspended particles such as other algae and inflow of non-point source pollutants, and to be highly useful for a more accurate carbon cycle mass balance assessment in a natural system containing particulate organic carbon (POC).

### BRIEF DESCRIPTION OF THE DRAWINGS

FIG. 1 shows a method of preparing a standard particle for quality control in a total organic carbon analysis according to one embodiment of the present disclosure;

FIG. 2 shows a diagram showing a particle size distribution of standard particles for each sieve particle size according to another embodiment of the present disclosure;

FIG. 3 shows a diagram showing the results of an interlaboratory comparison in Korea using standard particles prepared according to a further embodiment of the present disclosure;

FIG. 4 shows a diagram showing an example of standard particle samples stored in brown glass bottles, respectively, according to a yet further embodiment of the present disclosure.

### DETAILED DESCRIPTION

The present disclosure is to provide a standard particle suitable for quality control of suspended particles when analyzing total organic carbon for water samples containing the suspended particles. In particular, the present disclosure is to provide a standard particle with versatility which can be used for quality control for a total organic carbon analysis of water samples containing suspended particles by selecting and collecting target samples of an environmentally similar matrix from environmental samples, cooling and freeze-drying the target samples, and then homogenizing the target samples.

The standard particle according to the present disclosure may be used for reliable total organic carbon analysis procedures, condition settings, and proficiency evaluation such as sample handling, pretreatment, homogeneity evaluation, and device setup for water samples containing actual suspended particles.

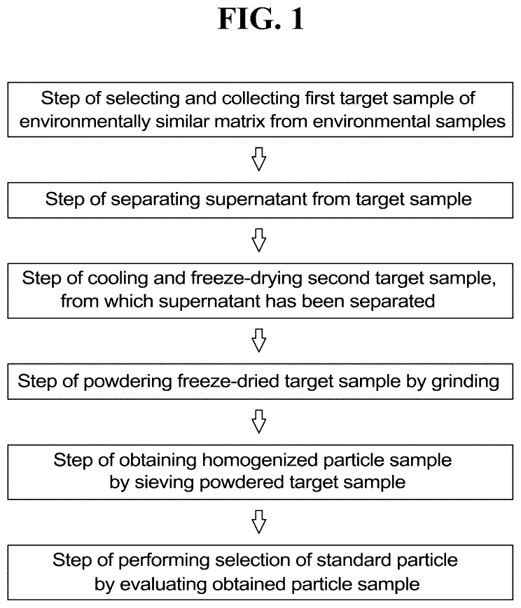

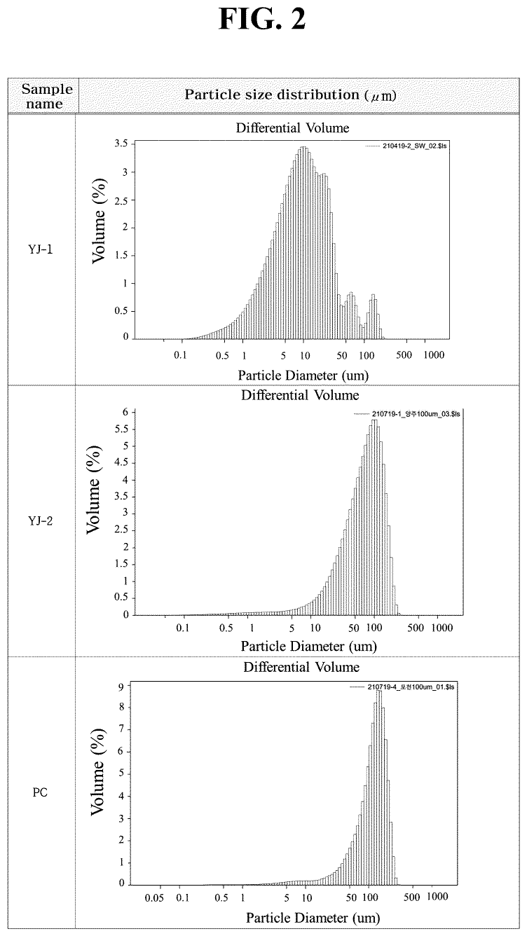

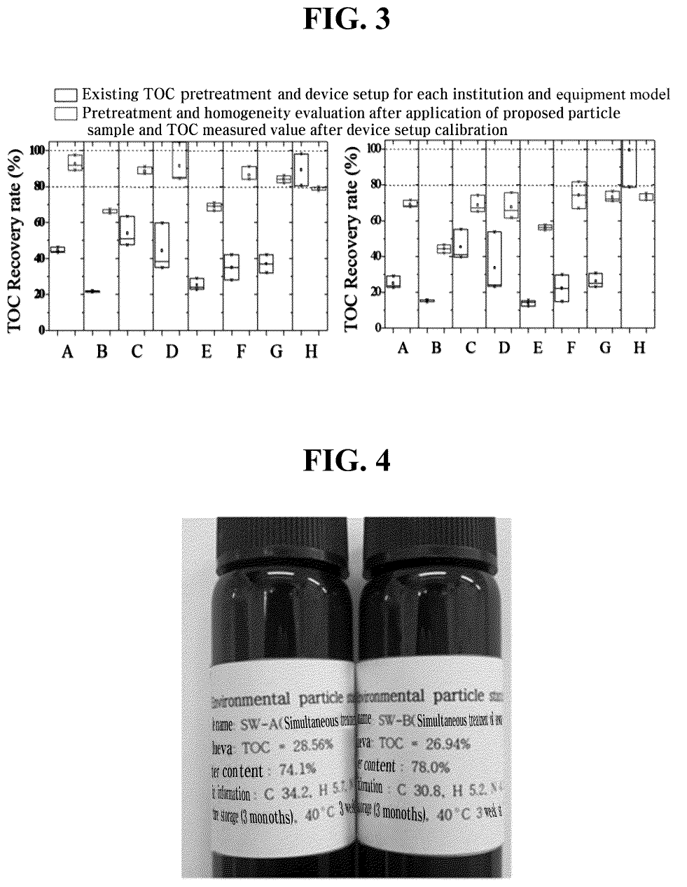

Hereinafter, embodiments of the present disclosure will be described with reference to the attached drawings. FIG. 1 shows a method of preparing a standard particle for quality control in a total organic carbon analysis according to one embodiment of the present disclosure. FIG. 2 shows a diagram showing a particle size distribution of standard particles for each sieve particle size according to another embodiment of the present disclosure. FIG. 3 shows a diagram showing the results of an interlaboratory comparison in Korea using standard particles prepared according to a further embodiment of the present disclosure. FIG. 4 shows a diagram showing an example of standard particle samples stored in brown glass bottles, respectively, according to a yet further embodiment of the present disclosure.

As shown in FIG. 1, the method of preparing a standard particle for quality control in a total organic carbon analysis according to the present disclosure includes: a step of selecting and collecting a target sample of an environmentally similar matrix from an environmental sample, a step of separating a supernatant from the target sample, a step of cooling and freeze-drying a target sample, from which the supernatant has been separated, a step of powdering the freeze-dried target sample by grinding, a step of obtaining a homogenized particle sample by sieving the powdered target sample, and a step of performing selection of a standard particle by evaluating the obtained particle sample.

### <Preparation of Standard Particle>

The present disclosure is intended to provide a standard particle suitable for quality control of suspended particles when analyzing total organic carbon (TOC) for water samples. First, target samples of an environmentally similar matrix are selected and collected from environmental samples.

The environmental samples may be selected in various ways considering the medium of the to-be-measured samples, such as algae, soil, livestock manure, and sewage sludge.

In one embodiment of the present disclosure, sewage sludge, which was a medium similar to the environment (the environmentally similar matrix), was used as the environmental samples to prepare particle samples of environmental origin suitable for sample analysis of wastewater influent, process water, and leachate. In particular, biologically stabilized and excess sewage sludge among the sewage sludge was used as the environmental samples. From the sewage sludge, the target samples were collected.

The reason for using sewage sludge as the target samples to prepare the standard particles in the one embodiment of the present disclosure was that the main target to become the water samples containing the suspended particles to measure total organic carbon was sewage and wastewater samples (environmental regulations on total organic carbon in wastewater discharge has begun in 2021), and the sewage and wastewater samples were of environmental origin, so the sewage and wastewater samples were likely to be suitable as standard materials.

Additionally, the sewage sludge (excess sludge) was a final sludge generated during a wastewater treatment process and had gone through a biological treatment process. Thus, it was likely that the sewage sludge contained organic matter in greater stability compared to influent water, and the homogeneity and stability of the standard particles were high due to minimization of elution of dissolved organic matter, and also the accuracy and precision of quality control might be improved.

Additionally, it was easy to secure a significant amount of samples by using the sewage sludge, and it was easy to separate solid and liquid through centrifugation due to high specific gravity of the sewage sludge. This made it easy to prepare particle samples, thereby offering an economic advantage.

The sewage sludge used in the present disclosure for collecting the target samples might be collected from various domestic wastewater treatment plants in Korea. The collection might occur at specially selected wastewater treatment plants considering treatment capacity, inflow sources, and treatment methods.

In another embodiment of the present disclosure, the target samples were collected directly through the excess sludge transfer pipe of the wastewater treatment plants. The target samples were collected in a volume of 20 L in a container made of high molecular high-density polyethylene (HDPE), and the collected target samples were immediately transported to the laboratory and stored at a temperature of 4° C.

In a further embodiment of the present disclosure, two representative domestic wastewater sources (Yangju: YJ-1, YJ-2) and one wastewater origin (Pocheon: PC) were selected to collect the target samples. In particular, since the wastewater originating from Yangju (YJ) had large treatment capacities and was wanted to be comparatively evaluated depending on collection time, samples (of wastewater originating from Yangju) were obtained in winter (YJ-1) and summer (YJ-2), respectively, and compared.

Table 1 below shows the basic information of the target samples collected from sewage sludge to prepare standard particles of the present disclosure.

TABLE 1

| | | Sites of basic | | | | | | |
|---|---|---|---|---|---|---|---|---|
| Sample | | environmental | Processing | | Influent properties | | | |
| classification | Influent | facilities | capacity | Treatment | SS | LOI | mg-C/L | |
| symbol | origin | (location) | (m³/L) | Method | (mg/L) | (%) | TOC | DOC |
| YJ-1 | Wastewater | 303, Gangbyeonseo- | 70,000 | DENSAEG + | 104.0 | 73.0 | 73.8 | 26.2 |
| | | ro, Eunhyeon-myeon, | | upstream | (75.9)^{a} | | | (35.5%)^{b} |
| | | Yangju-si (Sincheon | | biological | | | | |
| | | wastewater | | filtration | | | | |
| | | treatment plants) | | | | | | |
| | | (Collected in | | | | | | |
| | | February 2021) | | | | | | |
| YJ-2 | Wastewater | 303, Gangbyeonseo- | 70,000 | DENSAEG + | 185.0 | 83.3 | 89.0 | 39.9 |
| | | ro, Eunhyeon-myeon, | | upstream | (154.1) | | | (44.3%) |
| | | Yangju-si (Sincheon | | biological | | | | |
| | | wastewater | | filtration | | | | |
| | | treatment plants) | | | | | | |
| | | (Collected in | | | | | | |
| | | July 2021) | | | | | | |
| PC | Sewage | 17, Jungang-ro | 24,000 | Intermittent | 269.3 | 94.1 | 170.4 | 14.2 |
| | water | 461beon-gil, | | aeration | (253.1) | | | (8.3%) |
| | | Sinbuk-myeon, | | contact | | | | |
| | | Pocheon-si (Pocheon | | oxidation | | | | |
| | | Public wastewater | | method | | | | |
| | | treatment Facility) | | | | | | |
| | | (Collected in | | | | | | |
| | | July 2021) | | | | | | |

^{a}POM (mg/L) = SS (mg/L) × LOI (%).
^{b}Percentage of TOC.

A supernatant was separated from the target samples collected from the sewage sludge. The separation of the supernatant might preferably involve a first supernatant separation process by precipitation and a second supernatant separation process by centrifugation. By separating the supernatant from the raw sewage sludge, the stability of the target samples may be maintained, and an amount of dissolved organic matter may be minimized.

In the first supernatant separation process, the supernatant was first separated by immersion (natural sedimentation due to gravity) before being subjected to centrifugation. At this point, sediment and supernatant layers were confirmed with the naked eye, and the supernatant was separated using a pipette to prevent the sediment from being disturbed.

The first supernatant separation process was intended to shorten the separation time in the second centrifugation process. The first supernatant separation process was performed (about 1 to 2 hours) after settling until most of the suspended particles, except for fine particles in the supernatant, settle, and the supernatant and sediment layers were clearly visible. Usually, during the first immersion, about 11 L to 13 L of supernatant was removed out of a total 20 L sample containing suspended particles.

After the separation of the first supernatant by immersion, the first supernatant was secondarily put into a centrifuge to separate a second supernatant in the second time. Centrifugation was intended to shorten the freeze-drying time by lowering the moisture content of the sedimented samples.

In a yet further embodiment of the present disclosure, centrifugation was performed at 4000 rpm for 2 minutes using MF 600P Plus (Hanil Scientific Inc. MF-600 Plus). The degree of centrifugation might be appropriately performed depending on the specific gravity of a solution of the raw sewage sludge.

Then, the target samples, from which the second supernatant was separated, were cooled and freeze-dried. This was to dry the moisture while minimizing the effect on organic matter after the separation of the second supernatant.

In the case of the conventional hot air drying method, it was known that physical changes in the material were induced. Hence, porosity destruction, microstructure destruction, oxidation reaction, pH change, and ionic bond change occurred. In particular, loss of organic matter occurred. Due to that, it was preferable to proceed with freeze-drying rather than hot air drying.

In a still yet further embodiment of the present disclosure, cooling was performed in a refrigerator at a temperature in a range of 0° C. to −30° C., preferably −10° C. to −28° C., and more preferably −25° C. to freeze the target samples. Next, freeze-drying was performed using FD8508 (IlshinBioBase) at a temperature of −80° C. or lower and in 5 mTorr vacuum conditions.

In a still yet further embodiment of the present disclosure, the target samples refrigerated before freeze-drying might be frozen to a lower temperature using liquid nitrogen for faster drying if necessary. The freeze-drying was performed until the moisture in the target samples was completely removed. It varied depending on the moisture content of the target samples but took approximately 3 to 5 days.

Then, the freeze-dried target samples were ground into powder, and the powdered target samples were sieved to obtain homogenized particle samples.

The freeze-dried target samples were appropriately ground using a mortar or pestle. At this time, the use of powdering devices, such as mixers and grinders, was avoided because the powdering devices artificially reduced the particle size of the target samples. The main purpose of grinding was to separate the particles that had aggregated during the drying process.

The sieve might be used in various sizes depending on need. In a still yet further embodiment of the present disclosure, a sieve particle size was set to 100 μm such that the particle size of the standard particles according to the present disclosure became 100 μm or less. This procedure was performed to align the particle size with a particle size of cellulose standard particles (20 to 100 μm) used in domestic and international total organic carbon test methods and with a sieve particle size (<100 μm) for pretreatment used in sample homogenization applied in the domestic TOC-continuous monitoring method in Korea (KS; ES 04906.1d).

In a still yet further embodiment of the present disclosure, to homogenize the powdered samples, sieving (<100 μm) was performed using TEST SIEVE (Cheonggye), and then the powdered samples were homogeneously mixed for 24 to 48 hours using a shaking incubator (JSSI-100C, JSR).

To exclude the influence of microorganisms, the final homogenized samples went through a sterilization process (using a clean bench, 1 hour) through UV ray irradiation, and then the final homogenized samples were stored in a cool, dark place (4° C.) to block reactions caused by light.

To select suitable standard particles from the homogenized particle samples, a selection evaluation was performed. The selection evaluation might include evaluation items of homogeneity, stability, accuracy, and precision of the particle sample.

That is, according to the present disclosure, samples of various origins and particle sizes were screened to determine whether the samples were suitable for standard materials through homogeneity and stability evaluation. In addition, in the present disclosure, standard particles suitable for total organic carbon quality control were selected through accuracy and precision evaluation depending on sample pretreatment and device setup.

The standard particle samples prepared in this way were placed in a brown glass bottle, which did not transmit light, and stored in a cool and dark place. For interlaboratory testing, the prepared standard particle samples were placed in brown bottles, respectively, and delivered to each laboratory. FIG. 4 shows a diagram showing examples of standard particle samples stored in brown glass bottles, respectively, according to a yet further embodiment of the present disclosure.

### Homogeneity Evaluation Results

To obtain total carbon (TC) measurement (KS; ES 04862.1) and total organic carbon (TOC) measurement (KS; ES 04861.1) through elemental analysis of particle samples by using a sieve of a particle size of 100 μm according to the present disclosure, homogeneity and stability evaluations were conducted on the samples (Table 2 and Table 3). A homogeneity investigation method and result calculation were based on procedures specified in ISO 13528 (2000) and ISO Guide 35 (NIER, 2008).

That is, two test samples were taken from each of the 10 bottles into which the particle samples were added. Afterward, all test samples (test portion) were randomly measured under repeatability conditions to obtain measurement results for each. Analysis of variance was performed using these results. From the results, within-bottle homogeneity (S_{w}) and between-bottle homogeneity (S_{b}) were calculated in the following manner (NIER, 2008).

When data obtained from the homogeneity test was x_{t,k} [herein, t meant the particle samples (t=1, 2, . . . , nₛ), and k meant test samples (k=1, 2)]. An average of each sample (xₜ), a range between test samples (ωₜ), and an overall average (x̅) were defined as equations (1) to (3), respectively. A standard deviation of the samples meant (Sₓ), and the within-bottle homogeneity (S_{b}) were calculated as equations (4) to (6), respectively (NIER, 2008).

In the samples for proficiency testing, when the between-bottle homogeneity (S_{b}) satisfied the condition of ≤0.3σ_{PA} when compared with the standard deviation (σ_{PA}) to be used for proficiency evaluation, the samples were judged to have appropriate homogeneity.

The homogeneity evaluation was conducted with different standard deviations in accordance with KS standards and ISO standards. The KS standard used a standard deviation (σ_{PA}) of 20% as a quality control target, setting a target expanded uncertainty at 6. Meanwhile, the ISO standard used a standard deviation (σ_{PA}) of 10%, setting a target expanded uncertainty at 3 (ISO 8245, KS; ES 04311.1c).

$${x}_{t , .} = ( {x}_{t , 1} + {x}_{t , 2} ) / 2 \qquad ( 1 )$$

$${w}_{t} = \vert {x}_{t , 1} - {x}_{t , 2} \vert \qquad ( 2 )$$

$$\overline{x} = \sum x . , . / {n}_{S} \qquad ( 3 )$$

$${S}_{x} = \sqrt{\sum {( {x}_{t , .} - \overline{x} )}^{2} / ( {n}_{S} - 1 )} \qquad ( 4 )$$

$${S}_{W} = \sqrt{\sum {w}_{t}^{2} / 2 {n}_{s}} \qquad ( 5 )$$

$${S}_{b} = \sqrt{{S}_{x}^{2} - ( {S}_{W}^{2} / 2 )} \qquad ( 6 )$$

As a result of the homogeneity evaluation, in the total carbon (TC) case, the samples of all origins showed excellent homogeneity, and the samples of all origins were found to satisfy homogeneity of ISO and KS levels. In the total organic carbon case, the samples of all origins were also found to satisfy homogeneity of ISO and KS levels.

In other words, in terms of homogeneity, particle samples of all origins satisfied the levels suitable for proficiency evaluation suggested by domestic quality control standards. This indicated that the particle samples of all origins were suitable for serving as standard particles. In particular, the samples from the YJ-1 and PC satisfied homogeneity for the ISO quality control level. Certification values and standard uncertainties for each particle sample in the total carbon and total organic carbon cases are shown in Table 2 and Table 3, respectively.

Meanwhile, as a result of the homogeneity evaluation, it was confirmed that the between-bottle homogeneity of the samples of all origins decreased significantly in the total organic carbon case compared to the total carbon case. From this, it is believed that, in the total organic carbon case, during the removal of inorganic carbon through separate persulfuric acid treatment in the analysis process, coagulation and acid concentration were caused, which affected the homogeneity. Meanwhile, in the PC case, where its sample had a low content of inorganic carbon (less than 2%) among the samples, the decrease in homogeneity was not significant. From this, it is believed that a sample with a low content of inorganic carbon would have been more advantageous in securing the homogeneity of a particle sample.

TABLE 2

| | | | | Target | | | | | |
|---|---|---|---|---|---|---|---|---|---|
| | Sieve | Within- | Between- | extended | | | | TOC | |
| | particle | bottle | bottle | uncertainty | | Homogeneity | | certification | |
| Sample | size | homogeneity | homogeneity | (S_{b} ≤ 0.3 σ_{PA}) | | compliance | | value | Standard |
| name | (μm) | (S_{w}) | (S_{b}) | ISO | KS | ISO | KS | (%) | Uncertainty |
| YJ-1 | 100 | 0.295 | 0.146 | 3 | 6 | ∘ | ∘ | 34.2 | 0.029 |
| YJ-2 | 100 | 0.152 | 0.203 | 3 | 6 | ∘ | ∘ | 30.8 | 0.016 |
| PC | 100 | 0.029 | 0.026 | 3 | 6 | ∘ | ∘ | 37.2 | 0.007 |

TABLE 3

| | | | | Target | | | | | |
|---|---|---|---|---|---|---|---|---|---|
| | Sieve | Within- | Between- | extended | | | | TOC | |
| | particle | bottle | bottle | uncertainty | | Homogeneity | | certification | |
| Sample | size | homogeneity | homogeneity | (S_{b} ≤ 0.3 σ_{PA}) | | Compliance | | value | Standard |
| name | (μm) | (S_{w}) | (S_{b}) | ISO | KS | ISO | KS | (%) | Uncertainty |
| YJ-1 | 100 | 0.493 | 2.486 | 3 | 6 | ∘ | ∘ | 28.559 | 0.083 |
| YJ-2 | 100 | 0.662 | 2.683 | 3 | 6 | ∘ | ∘ | 26.939 | 0.430 |
| PC | 100 | 0.307 | 0.389 | 3 | 6 | ∘ | ∘ | 37.315 | 0.003 |

### Stability Evaluation Results

The stability evaluation according to a still yet further embodiment of the present disclosure was for evaluating the analyst's proficiency level when analyzing total organic carbon for water samples. To check the transport and storage conditions of the samples, especially for overseas transport, stability was evaluated for 2 to 4 weeks assuming that the samples were stored at a temperature of 40° C. under harsh conditions.

That is, the particle samples were stored in the dark at a temperature of 40° C. for two weeks and then used as samples to investigate stability in comparison with the samples stored at a temperature of 4° C. An element-by-element analysis was performed using the same method used for CRM (certified reference material) authentication. The measurement results of the samples, which had been stored at a temperature of 4° C. and 40° C., respectively, were compared.

Out of four samples, an average and coefficient of variation (CV) of the measurement results represented X̅_{T} and CV_{T} for two samples stored at a temperature of 4° C., and represented X̅_{40°} and CV_{40°} for two samples stored at a temperature of 40° C., respectively. In this situation, the ratio (R_{T}) of the average values in the two conditions was calculated as in Equation (7). Uncertainty (U_{T}) depending on temperature was obtained using Equation (8) with the ratio of the average values (R_{T}) and the CVs of the four samples, as obtained at each temperature.

When a sample was stable, in the ideal case, R_{T} was 1, and when the value R_{T} was in a range of (R_{T}+U_{T}) and (R_{T}−U_{T}), including 1 in the relevant storage period, the sample was considered stable because there was no significant change until the storage period.

$${R}_{T} = {\overline{X}}_{T} / {\overline{X}}_{40 ° C .} \qquad ( 7 )$$

$${U}_{T} = {( {CV}_{T}^{2} + {CV}_{40 ° C .}^{2} )}^{1 / 2} \cdot {R}_{T} \qquad ( 8 )$$

As a result of the stability evaluation, the short-term stability evaluation results of the particle samples under harsh transportation conditions are shown in Tables 4 and 5 below. It was confirmed that stability was mostly obtained in the second week in the total carbon and total organic carbon cases. When the evaluation was conducted for 3 weeks or more, stability dropped sharply, and most samples did not obtain stability. Therefore, the stability of the prepared particle samples was determined by a storage period of 2 weeks under harsh conditions.

TABLE 4

| | Sieve | | | | | | |
|---|---|---|---|---|---|---|---|
| Sample | particle | Week 2 | | | Week 3 | | |
| name | size (μm) | R_{T} | U_{RT} | Results | R_{T} | U_{RT} | Results |
| YJ-1 | 100 | 1.000 | 0.025 | Stable | 1.026 | 0.013 | Unstable |
| YJ-2 | 100 | 0.997 | 0.009 | Stable | 1.039 | 0.008 | Unstable |
| PC | 100 | 0.995 | 0.005 | Stable | 1.039 | 0.001 | Unstable |

TABLE 5

| | Sieve | | | | | | |
|---|---|---|---|---|---|---|---|
| Sample | particle | Week 2 | | | Week 3 | | |
| name | size (μm) | R_{T} | U_{RT} | Results | R_{T} | U_{RT} | Results |
| YJ-1 | 100 | 1.002 | 0.068 | Stable | 0.921 | 0.094 | Stable |
| YJ-2 | 100 | 0.969 | 0.049 | Stable | 1.076 | 0.041 | Unstable |
| PC | 100 | 0.996 | 0.011 | Stable | 0.983 | 0.012 | Unstable |

### Suitability for Proficiency Evaluation, Such as in Sample Homogenization Pretreatment and Device Setup

Based on the homogeneity and stability evaluation results above, standard solutions were prepared using the standard particles selected from the YJ-2 and PC particle samples, respectively. The standard solutions were subjected to quality control by the main pretreatment and device setup used in total organic carbon analysis. The standard solutions were prepared to have a concentration (mg/L) of 250 mg/L (ranging from 50 to 80 mg-C/L depending on origin). The concentration was similar to that of cellulose (225 mg/L, equivalent to 100 mg-C/L), which was a particle sample for quality control of suspended materials in the existing official test method (ISO 8245, KS; ES 04311.1c).

When analyzing total organic carbon for the standard solutions, a high-temperature combustion oxidation method (KS; ES 04311.1c) was used. For homogeneity, a turbidity index (NTU₅/NTU₀) was applied. Herein, the NTU₅/NTU₀ represented a turbidity ratio after 5 minutes compared to an initial turbidity at 0 minutes, following flotation. The closer the ratio value was to 1, the higher the homogeneity of the sample by pretreatment was evaluated.

As a result of the analysis, the recovery rate and precision were found to be different depending on the pretreatment method, sieving, and whether or not the resuspension mode was applied. Thus, it is expected that it would be possible to evaluate the analyst's proficiency in sample handling and measurement (Table 6, Table 7, and Table 8).

Regarding pretreatment methods, improvements in the recovery rate and precision, as well as the effect of sieving the pretreated samples were evaluated by simple stirring (Stir) and an ultrasonic pretreatment method (US), which were suggested by the existing official test method, and an combined ultrasonic and alkaline extraction pretreatment (CULA) method secured by our research team for the present disclosure, respectively.

Regarding resuspension mode, due to improvement in particle sedimentation, a reduction level in the recovery rate and precision was evaluated. In the evaluation, the difference in whether resuspension was possible depending on the current total organic carbon analysis device type was taken into account.

Regarding homogeneity evaluation, a turbidity index showed a high correlation with precision. Thus, it was possible to evaluate the effectiveness of sample pretreatment and resuspension mode application by homogeneity evaluation using turbidity indicators.

In this way, the precision (RSD, %) of the standard particle samples suitable for proficiency evaluation for selecting appropriate standard particles needs to satisfy the KS standard of <20%. The recovery rate needs to be guaranteed at a minimum level that allows comparative evaluation by sample handling and device setup (resuspension mode).

In a still yet further present disclosure, total organic carbon analysis was performed on the standard solutions after preparation of the standard solutions using the standard particles. An evaluation was performed by setting the recovery rate to 50% or more (This value was arbitrarily selected to ensure the reliability of recovery rate evaluation.) after ultrasonic homogenization pretreatment (including application of resuspension mode), which was widely used in the current official test method and by setting the recovery rate to 80% or more as standards suitable for selecting standard particles when applying the new pretreatment method (CULA).

As a result of the evaluation, in the case of a sieve particle size of 500 μm and sample particle size of 2000 μm, the samples were unsuitable for use as quality control particles because the samples did not satisfy the standards (data not shown). Meanwhile, in experiments with a sieve particle size of 100 μm or less, the samples showed differences in the recovery rate and precision depending on pretreatment and resuspension, so the samples were suitable for use as proficiency evaluation samples. In particular, the sample from the YJ-2 had sample characteristics of a high CULA pretreatment effect, so the sample from the YJ-2 was suitable for proficiency evaluation by alkaline pretreatment. The sample from the PC showed a high recovery rate even under simple stirring or ultrasonic treatment effects, so the sample from the PC might be usefully used for proficiency evaluation in current official testing methods (KS, ISO). The sample from the YJ-1 also showed similar results to that of the YJ-2 (Table 6 and Table 7).
[symbol] KS standard (RSD<20%, recovery rate >80%)
[symbol] ISO standard (RSD<10%, recovery rate >90%)

### Selection of Optimal Standard Particle Samples and Physical and Chemical Properties of Particles

As a result of the homogeneity and stability evaluation of the particle samples according to the present disclosure, all samples satisfied the target standard deviation of domestic standard in Korea. In particular, the samples from the YJ-1 and PCalso satisfied international standards (ISO).

As a result short-term stability evaluation under harsh conditions considering the transportation process when used as a proficiency evaluation sample, most samples did not obtain the stability after the 3rd week. Thus, the storage period of particle samples according to the present disclosure was 2 weeks from the date of sample shipment.

Considering the homogeneity evaluation results obtained, the sample from the YJ-1 was suitable for domestic and international proficiency evaluation, and the sample from the YJ-2 was suitable as a domestic proficiency evaluation sample. The physicochemical property information of the standard particle samples is shown in Table 9 and FIG. 2.

TABLE 9

| | | | Loss on | | | | |
|---|---|---|---|---|---|---|---|
| | Density | Zeta | ignition | Elemental analysis (%) | | | |
| Sample name | (g/mL) | Potential | (LOI, %) | N | C | H | S |
| YJ-1 | 1.65 | −12.06 | 70.0 | 4.4 | 34.2 | 5.6 | 0.7 |
| YJ-2 | 1.76 | −17.7 | 7777- | 4.2 | 30.8 | 5.2 | 1.4 |
| PC | 1.57 | −22.4 | 80.0 | 7.4 | 37.5 | 6.1 | 0.9 |

### <Verification Through Interlaboratory Comparison>

Interlaboratory comparison evaluation among domestic laboratories was conducted on the samples from the YJ-2 and PC, the samples having been derived on the basis of standard particles selected after completion of suitability evaluation according to the present disclosure.

All of the institutions (8 locations) subject to the Interlaboratory comparison were those that satisfied the conventional cellulose quality control conditions. In the institutions, Interlaboratory comparison were conducted to compare and evaluate the analyst's proficiency in analyzing standard particle samples according to the present disclosure.

As a result of the analysis, in the sample from the YJ-2, a 45.7% occurred depending on the analyst and reproducibility deviation equipment model. The sample also showed a recovery rate with a large difference, ranging from 30% to 90%. In the sample from the PC, differences in the reproducibility deviation and recovery rate were 77.21% and 18% to 80%, respectively (Table 10).

This means that even when the institutions satisfied the conventional cellulose quality control conditions, there was a large deviation in the analysis value depending on the analyst's proficiency level when handling actual environmental samples, as a result, practical quality control was not implemented.

In the experiments, analytical procedure know-how for application of chemical pretreatment (CULA) technique (Refer to Korean Patent Application Publication No. 10-2020-0119794) obtained in the present inventors' laboratory and for device setup to analyze samples containing suspended materials was additionally delivered. By doing so, quality control using the standard particle samples according to the present disclosure was additionally performed and the results were compared.

As a result, reproducibility was greatly improved from 45.7% to 12.8% for the sample from the YJ-2 and from 77.2% to 16.4% for the sample from the PC. The recovery rate (%) was also greatly improved from 41.9 to 82.3 and 32.4 to 65.5 for the samples of the YJ-2 and PC, respectively, and the variation among laboratories was also greatly reduced (Table 10 and FIG. 3).

In other words, it was possible to significantly improve the reproducibility and recovery rate of measured values in quality control for sample pretreatment, homogeneity evaluation, and device setup using standard particles according to the present disclosure.

TABLE 10

| Quality | Sample | | | O | X | =x | n | Sᵣ | C_{V.R} | Sᵣ | C_{V.r} |
|---|---|---|---|---|---|---|---|---|---|---|---|
| control | name | l | n | % | mg/l | mg/l | % | mg/l | % | mg/l | % |
| Conventional | YJ-2 | 8 | 23 | 11.54 | 14.28 | 5.98 | 41.88 | 1.18 | 15.37 | 2.74 | 45.74 |
| method | PC | 8 | 23 | 11.54 | 13.47 | 4.36 | 32.37 | 1.15 | 18.86 | 3.37 | 77.21 |
| After | YJ-2 | 8 | 23 | 11.54 | 14.28 | 11.75 | 82.28 | 0.32 | 2.54 | 1.50 | 12.77 |
| quality | PC | 8 | 23 | 11.54 | 13.47 | 8.82 | 65.48 | 0.64 | 6.90 | 1.44 | 16.38 |
| control | | | | | | | | | | | |
| application | | | | | | | | | | | |

l: number of laboratories after outlier rejection (Number of institutions subject to interlaboratory comparison)
n: number of individual test results after outlier rejection
O: percentage of outliers
X: assigned value
=, x: overall mean of results (without outliers)
n: recovery rate
Sᵣ: reproducibility standard deviation
C_{V.R}: coefficient of variation of reproducibility
Sᵣ: repeatability standard deviation
C_{V.r}: coefficient of variation of repeatability

As described above, the present disclosure is to provide standard particles suitable for quality control of suspended particles when analyzing total organic carbon for water samples. In particular, the present disclosure is to provide standard particles with versatility which may be used for quality control for a total organic carbon analysis of water samples containing suspended particles by collecting target samples from sewage sludge, cooling and freeze-drying the target samples, and then homogenizing the target samples.

In addition, the standard particles of the present disclosure are selected as standard particles suitable for quality control of total organic carbon by screening samples suitable for standard materials through homogeneity and stability evaluation and evaluating accuracy and precision depending on sample preparation and device setup.

In addition, the effectiveness of the TOC equipment model and quality control method has been verified through Interlaboratory comparison at domestic testing institutes using the standard particles selected according to the present disclosure.

Thus, the standard particle according to the present disclosure may be used for reliable total organic carbon analysis procedures, condition settings, and proficiency evaluation such as sample handling, pretreatment, homogeneity evaluation, and device setup for water samples containing actual suspended particles. This allows practical quality control and improves the wastewater treatment process.

In addition, the standard particles according to the present disclosure are prepared from sewage sludge (excess sludge) which has completely undergone a biological-based wastewater treatment process, thereby the standard particles have the properties of particulate organic matter with low biodegradability, so the standard particles are highly stable and may be secured in large quantities. This creates a high added value of organic waste through recycling of excess sludge, making an economic effect.

In addition, it is generally important to accurately measure total organic carbon in wastewater influent in predicting an organic matter treatment rate for each unit process. By using the standard particles according to the present disclosure, the accurate measurement of total organic carbon for each unit process is possible. Thus, in the calculation of chemical dosage and processing time during the treatment process, a significant improvement may be achieved. Accordingly, the standard particles according to the present disclosure are expected to have a high potential for use in quality control to measure total organic carbon in wastewater treatment plants with a high possibility of inflow of suspended particles.

In addition, the present disclosure is expected to be highly useful for an accurate measurement evaluation of total organic carbon for river samples, which are affected by suspended particles such as other algae and inflow of non-point source pollutants, and to be highly useful for a more accurate carbon cycle mass balance assessment in a natural system containing particulate organic carbon (POC).

1. A method of preparing a standard particle for quality control in a total organic carbon analysis, the method comprising:
a step of selecting and collecting a target sample of an environmentally similar matrix from an environmental sample,
a step of separating a supernatant from the target sample,
a step of cooling and freeze-drying a target sample, from which the supernatant has been separated,
a step of powdering the freeze-dried target sample by grinding,
a step of obtaining a homogenized particle sample by sieving the powdered target sample, and
a step of performing selection of a standard particle by evaluating the obtained particle sample.

2. The method of claim 1, wherein the standard particle has a particle size in a range of 100 μm or less.

3. The method of claim 1, wherein the step of separating the supernatant comprises a first supernatant separation process by precipitation and a second supernatant separation process by centrifugation.

4. The method of claim 1, wherein the step of cooling and freeze-drying further comprises a cooling process using liquid nitrogen.

5. The method of claim 1, wherein the step of obtaining the particle sample further comprises mixing by shaking for 24 to 48 hours after the sieving.

6. The method of claim 1, wherein the step of obtaining the particle sample further comprises a sterilization process through UV ray irradiation after the sieving.

7. The method of claim 1, wherein the step of performing selection of a standard particle comprises evaluating homogeneity, stability, accuracy, and precision of the particle sample.

8. The method of claim 7, wherein, in the homogeneity evaluation, a between-bottle homogeneity (S_{b}) satisfies the condition of S_{b}≤0.3σ_{PA} when compared to the standard deviation (σ_{PA}) to be used in the proficiency evaluation, and the between-bottle homogeneity (S_{b}) satisfies S_{b}≤3 to 6 (with a standard deviation of 10% to 20% applied in this range).

9. The method of claim 7, wherein the homogeneity evaluation is performed by applying a turbidity index (NTU₅/NTU₀) (wherein the NTU₅/NTU₀ represents a turbidity ratio after 5 minutes compared to an initial turbidity at 0 minutes, following flotation).

10. The method of claim 1, wherein, in the step of performing selection of the standard particle, a recovery rate of 50% to 80% or more is set as an appropriate standard for selecting the standard particle when preparing a standard solution using the standard particle and then performing the total organic carbon analysis on the standard solution.

11. A standard particle prepared by the method of claim 1 for quality control in a total organic carbon analysis of a sample of environmental origin.
